(19) United States
(12) Patent Application Publication
(10) Pub. No.: US 2022/0168529 A1
(43) Pub. Date: Jun. 2, 2022

(54) DOUBLE CUFF, SINGLE-LUMEN ENDOTRACHEAL TUBE FOR LUNG ISOLATION AND PROTECTION

(71) Applicant: University of Florida Research Foundation, Inc., Gainesville, FL (US)

(72) Inventor: Michael Edward Franklin, Gainesville, FL (US)

(21) Appl. No.: 17/441,560

(22) PCT Filed: Mar. 26, 2020
(86) PCT No.: PCT/US2020/024903

§ 371 Date: Sep. 21, 2021

Related U.S. Application Data
(60) Provisional application No. 62/823,835, filed on Mar. 26, 2019.

(51) Int. Cl. A61M 16/04; A61M 16/08; A61B 1/267

(52) U.S. Cl. CPC A61M 16/0459; A61M 16/0486; A61M 16/0816; A61M 2205/583; A61M 16/0479; A61B 1/2676; A61M 2205/32; A61M 16/0404

(57) ABSTRACT

Various embodiments of a double cuff single lumen endotracheal tube device for lung isolation and protection and its method of use are described herein. A device for intubation providing for single lung isolation and a transition to ventilation of both lungs is described. In one example, the device can be used extend into a main bronchus of patient. A distal cuff and secondary cuff can be inflated for ventilation of one lung. An auxiliary conduit provides passive oxygenation to the non-ventilated or alternately evacuation of mucus or fluids above the distal cuff. The device can be repositioned for long term intubation and ventilation of both lungs.

### CROSS-REFERENCE TO RELATED APPLICATION

This application claims the benefit of and priority to U.S. Provisional Application No. 62/823,835, titled “DOUBLE CUFF, SINGLE-LUMEN ENDOTRACHEAL TUBE FOR LUNG ISOLATION AND PROTECTION,” filed on Mar. 26, 2019, the contents of which are hereby incorporated herein by reference in its entirely.

### BACKGROUND

An endotracheal tube is a catheter that is inserted into the trachea for the primary purpose of establishing and maintaining a patent airway and to ensure the adequate exchange of oxygen and carbon dioxide. Endotracheal tubes are commonly used for airway management in the settings of general anesthesia, critical care, mechanical ventilation, and emergency medicine. However, a standard endotracheal tube is not effective in the cases where lung isolation or one-lung ventilation is indicated.

### SUMMARY

One embodiment includes an intubation device for single or double lung ventilation. The intubation device includes a flexible tubular body having a wall extending a length from a proximal end to a distal end forming a main lumen with an inner diameter, a distal portion having an bevel tip forming an opening to the main lumen, the bevel tip formed at the distal end with a tapered angle relative to a longitudinal axis of the flexible tubular body and a distal aperture formed in the wall opposite the opening. The device also includes a distal cuff may include a tubular inflatable material, the distal cuff attached at the distal portion of the flexible tubular body above the bevel tip and the distal aperture. The device also includes a secondary cuff may include a tubular inflatable material, the secondary cuff attached at a predetermined distance from the bevel tip of the flexible tubular body. The device also includes an auxiliary conduit may include an auxiliary channel extending from a proximal portion of the flexible tubular body to an auxiliary aperture positioned between the distal cuff and secondary cuff and an auxiliary tube sealingly connected to the auxiliary channel in the wall at the proximal portion of the flexible tubular body. The device also includes a main proximal connector connected to the proximal end of the flexible tubular body, the main proximal connector configured to detachably connect to a mechanical ventilation device.

Implementations may include one or more of the following features. The intubation device where: a first port is sealing attached to the first tube, the first port having a first valve configured to receive a gas provided to inflate the distal cuff; and a second port is sealing attached to the second tube, the second port having a second valve configured to receive a gas provided to inflate the secondary cuff. A third port is sealing attached to the third tube, the third port configured to receive at least one of: a detachable cap, a connection to a suction machine, and a connection to a continuous positive airway pressure (CPAP) machine.

The intubation device may include a first indicator that circumscribes the flexible tubular body, the first indicator located at the distal portion and positioned between the distal cuff and the auxiliary aperture, the first indicator configured to mark a first longitudinal distance from the proximal end of the flexible tubular body. The intubation device may include a second indicator that circumscribes the flexible tubular body, the second indicator positioned between the proximal end and the secondary cuff, the second indicator configured to mark a second longitudinal distance from the proximal end of the flexible tubular body. The distal portion of the intubation device is configured to extend into a bronchus of a patient. The distal portion of the intubation device is configured to be positioned in a trachea of a patient to provide ventilation to both lungs. The inner diameter of the main lumen is configured to slidingly receive a fiber optic bronchoscope (FOB). The intubation device may include a least one radio-opaque indicator configured to mark the position of the radio-opaque indicator on a radiograph to confirm proper positioning of the intubation device.

Another embodiment includes an intubation method for lung isolation and protection. The intubation method also includes using a fiber optic bronchoscope to guide a bevel tip of a distal end of an intubation device into a mainstem bronchus of a patient through the trachea. The intubation method also includes confirming the position of the distal end of an intubation device in a bronchus through the trachea of a patient. The intubation method also includes inflating a distal cuff to secure the bevel tip in the bronchus for single lung ventilation. The intubation method also includes inflating a secondary cuff to protect a non-ventilated lung from aspiration.

The intubation method may include delivering continuous positive airway pressure to the non-ventilated lung via an auxiliary channel and an auxiliary aperture within a wall of the intubation device. The intubation method may include suctioning secretions via an auxiliary aperture within the wall of the intubation device. The intubation method may include deflating the distal cuff; deflating the secondary cuff; repositioning the intubation device by withdrawing the bevel tip from the bronchus into the trachea without fully extubating; confirming the position of the bevel tip in the trachea; inflating the distal cuff in the trachea; and ventilating both lungs.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 illustrates an example device for a double cuff single lumen endotracheal tube with both cuffs deflated according to various embodiments described herein.

FIG. 2 illustrates an example device for a double cuff single lumen endotracheal tube with both cuffs inflated according to various embodiments described herein.

FIGS. 3A-3C illustrate example cross sections of the device at different longitudinal distances indicated on FIG. 2 according to various embodiments described herein.

FIG. 4 illustrates an example illustration of placement of a double cuff single lumen endotracheal tube in a bronchus according to various embodiments described herein.

FIG. 5 illustrates an example illustration of placement of a double cuff single lumen endotracheal tube in a trachea according to various embodiments described herein.

The drawings illustrate only example embodiments and are therefore not to be considered limiting of the scope of the embodiments described herein, as other embodiments are within the scope of this disclosure. The elements and features shown in the drawings are not necessarily drawn to scale, emphasis instead being placed upon clearly illustrating the principles of the embodiments. Additionally, certain dimensions or positionings may be exaggerated to help visually convey certain principles. In the drawings, similar reference numerals between figures designate like or corresponding, but not necessarily the same, elements.

### DETAILED DESCRIPTION

The present disclosure relates to a double cuff single lumen tube device for lung isolation and protection. The double cuff single lumen tube device (also referred to as the device herein) and method of use are described. The device can be used in medical procedures requiring ventilation of one lung, ventilation of both lungs, or ventilation of one lung and passive oxygenation of the other lung.

As noted above, an endotracheal tube (ETT) is commonly used for airway management in the settings of general anesthesia, critical care, mechanical ventilation, and emergency medicine to establish and maintain a patent airway and to ensure the adequate exchange of oxygen and carbon dioxide. However, a standard endotracheal tube is not effective in the cases where lung isolation or one-lung ventilation is indicated. Bronchial blockers or double lumen tubes are currently the most commonly used devices for lung isolation.

A bronchial blocker can be deployed to block the bronchus of one lung and allow ventilation of the other lung. The bronchial blocker device can be placed by guiding the device through a standard ETT. However, the bronchial blocker can be difficult to place and can become dislodged. While this combination may allow extended intubation after a procedure without replacing the tube, it is not ideal for many circumstances.

More commonly, a double lumen tube (DLT) is used for lung isolation, allowing ventilation to one or both lungs. A DLT provides two distinct lumens to deliver air: a tracheal lumen and a bronchial lumen. A DLT can selectively ventilate one lung or the other by moving a clamp from one point to the next and opening a valve. For example, in the case of one lung ventilation, once the DLT is placed in a left main stem bronchus, the bronchial cuff can be inflated, and the tracheal lumen clamped off so no air passes to the right lung. Because each lumen is sized to allow active ventilation, the DLT can be large in diameter, making it more difficult to insert and place. The DLT can additionally cause airway trauma and/or bleeding due to size. Moreover, a tube exchange is usually required for patients that require prolonged ventilation after surgery.

In contrast, the double cuff single lumen tube device and its method of use described herein can be easier to place and is better for long term ventilation, with not as much pressure on the airway. The double cuff single lumen tube device can be smaller in diameter than a DLT, because only one lumen is actively used for ventilation. For example, the device would be advantageous in difficult airway cases requiring lung isolation, allowing easier placement than a DLT or a bronchial blocker. The device protects the non-ventilated lung from aspiration better than simply placing a standard ETT in the main stem bronchus of the contralateral lung. Additionally, the device can be useful in patients requiring lung isolation for an extended period of time, such as broncho-pleural fistula patients requiring mechanical ventilation in the ICU.

Furthermore, the device can be useful in situations where a DLT would be difficult or undesirable to place. For example, in thoracoabdominal aortic cases, when a DLT placed for lung isolation must be exchanged for a single lumen ETT at the end of the case, there is risk involved with the tube exchange because the patient can be quite edematous. Similarly, for a mini-mitral valve repair, lung isolation is needed, but the patient remains intubated for a significant period of time. The double cuff single lumen tube device described herein would be preferable to leaving a large double lumen tube in place, because the device could simply be partially withdrawn into the trachea and used to ventilate both lungs. The device can be repositioned by deflating both cuffs, retracting the tube into the trachea, then re-inflating the distal cuff to ventilate both lungs.

The double cuff single lumen tube device may also be useful in smaller or pediatric patients where a very small double lumen tube would be normally indicated. In such cases, small double lumen tubes are of limited usefulness due to the availability of fiber optic bronchoscope equipment that can be used to verify positioning. The lumens of some smaller DLT are too small to easily use most bronchoscopy equipment to confirm position. However, a standard fiber optic bronchoscope could easily be placed down double cuff single lumen tube device to aid in positioning. Further, the device may have a lower likelihood of iatrogenic damage to the tracheobronchial tree than with bronchial blocker or EZ blocker placement. The device may provide better lung isolation than with a bronchial blocker and can be easier to place than a bronchial blocker, particularly when ventilating only the left lung.

In the context outlined above, the embodiments described herein are directed to a double cuff single lumen endotracheal tube device for lung isolation and protection. The double cuff single lumen tube device provides an alternative to conventional ventilation techniques and devices for lung isolation, including a double lumen endotracheal tube, a bronchial blocker, and other devices and techniques. The device is more cost effective, less invasive, and easier to use than other devices available on the market.

The double cuff single lumen endotracheal tube device described herein, provides for ease of placement of single lumen tube. The device can be used for either one lung or two lung ventilation. The device a can also be used for prolonged ventilation without a tube exchange.

The device includes a distal inflatable cuff is near the bevel tip of the device beyond which is a larger slotted opening, as with a standard single-lumen ETT. The bevel tip of the device can be placed in either the trachea or, with the aid of a flexible fiberscope, either mainstem bronchus of the lungs of a patient. A secondary inflatable cuff positioned several centimeters higher on the device than the distal cuff can be configured to protect the non-ventilated lung from aspiration by inflating the secondary cuff in the trachea.

When used for one-lung ventilation, the double cuff single lumen endotracheal tube device provides a main proximal connector for ventilation via a single lumen to the lung of interest through a bevel tip open at the distal end of lumen. The distal end of the lumen also comprises a fenestration in the wall inside of the tube, commonly known as a “murphy eye,” as an alternative vent if the main tube becomes clogged.

An auxiliary channel runs longitudinally within the wall of the double cuff single lumen endotracheal tube device with an aperture in the wall on the distal end that is located between the proximal cuff and distal cuff. The auxiliary channel can have a removably capped port on the proximal end. In an embodiment, if the device is placed in the mainstem bronchus for lung isolation, the auxiliary channel can be used to deliver passive oxygenation to the non-ventilated lung. For example, 5 cm of water for continuous positive airway pressure (CPAP). However, the auxiliary channel is relatively small and might not be suitable for meaningful ventilation in every embodiment. The capped port is not necessarily configured or intended to be used for mechanical ventilation of the second lung. When the device is placed in the trachea to ventilate both lungs, the auxiliary channel can also be used for suction or evacuation of mucus, secretions, blood, or other fluids simultaneously while venting the single lung.

In an embodiment, the distal cuff can be configured to occlude either bronchus or the trachea, depending on where it is positioned. In an embodiment, the device comprises an indicator marker to be visible during direct laryngoscopy. For example, the indicator marker can be a red mark around the outside of the device, approximately 3 cm above each cuff.

The device can be sized and configured based on the internal diameter (ID) of the main lumen, with measurements similar in range as a standard hi-low evac single lumen ETT. For example, the device can be configured with an internal diameter of about 2.0 mm to about 10.5 mm for the main lumen, being sized in 0.5 mm increments. The outer diameter (OD) can correspond to the configuration of the internal diameter, ranging from about 4.0 mm to about 13.5 mm. The distal cuff and proximal cuff on a device can be similar in size with an inflated diameter corresponding to the size of the device and ranging from about 20.0 mm to about 33 mm. The length of the device can be slightly longer than a standard ETT, ranging in from about 340 mm to about 420 mm, depending on the configuration.

In some embodiments, the device can have some features common to a standard ETT, such as a radio-opaque stripe, 15 mm ventilator/circuit attachment, and numerical depth markers. The device can be used with BODAI swivel or similar device for bronchoscopy. In one example, visual indicators are provided 3 cm off each cuff to aid in placement while advancing and withdrawing the device into trachea.

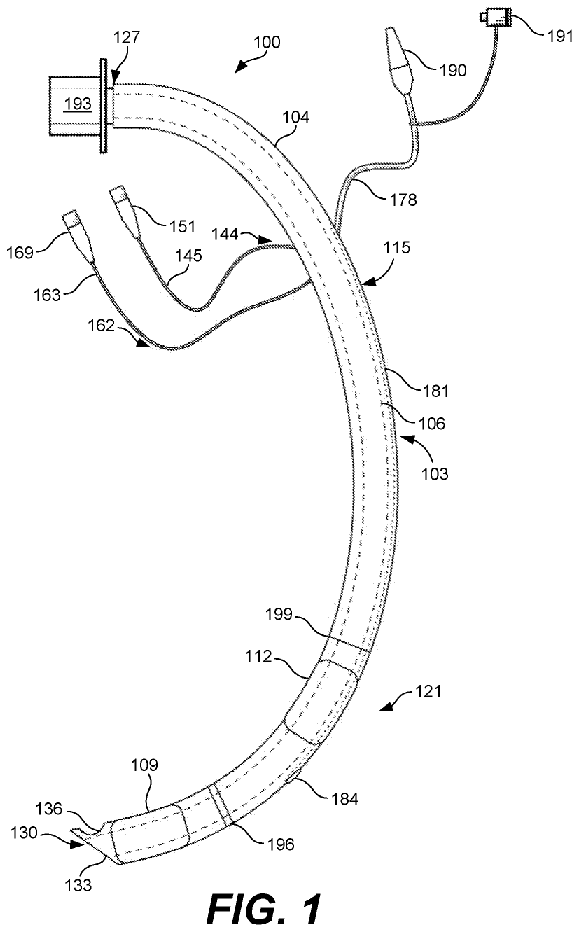

Turning to the drawings, FIG. 1 illustrates an example of an intubation device 100 for single or double lung ventilation. The intubation device 100 includes a flexible tubular body 103 having a main lumen 106, a distal cuff 109, a secondary cuff 112, and an auxiliary conduit 115. The intubation device 100 includes a proximal portion 118 and a distal portion 121. The main lumen 106 can be configured with an inner diameter (ID) configured to slidingly receive a fiber optic bronchoscope (FOB) to facilitate placement of the device. In this example, the device, although not shown to scale, is shown with a curvature to facilitate the placement of the device in either the trachea or bronchus of a patient.

The flexible tubular body 103 includes a wall 104 extending a longitudinal length (L) from a proximal end 127 to a distal end 130 forming the main lumen 106 with an inner diameter (ID). A distal portion 121 of the flexible tubular body 103 includes a bevel tip 133 open to the main lumen 106. The bevel tip 133 can be formed at the distal end 130 with a tapered angle relative to a longitudinal axis of the flexible tubular body 103. In an embodiment, a distal aperture 136 (also referred to as a Murphy eye herein) can be formed in the wall 104 opposite the bevel tip 133.

The distal cuff 109 can be formed from a tubular inflatable material. Shown as deflated in FIG. 1, the distal cuff 109 can be attached towards the distal portion 121 of the flexible tubular body 103 above the bevel tip 133 and the distal aperture 136. The distal cuff 109 can be inflated or deflated via a first conduit 144 in fluid communication with the distal cuff 109. The first conduit 144 comprises a first tube 145 sealingly connected to a first channel 146 (not shown) in the wall 104 of the flexible tubular body 103. The first conduit 144 further comprises a first port 151 sealing attached to the first tube 145. The first port 151 having a first valve configured to receive a gas provided to inflate the distal cuff 109. In an embodiment, the distal cuff 109 can be deflated during the positioning of the intubation device 100 in the trachea or bronchus of a patient. Although the first channel 146 is not shown, it will be discussed in further detail.

The secondary cuff 112 can be formed from a tubular inflatable material. As shown, the secondary cuff 112 can be attached at a predetermined distance from the bevel tip 133 of the flexible tubular body 103. The secondary cuff 112 can be inflated or deflated via a second conduit 162 in fluid communication with the secondary cuff 112. The second conduit comprises a second tube 163 sealingly connected to a second channel 164 (not shown) in the wall 104 of the flexible tubular body 103. The second conduit 162 further comprises a second port 169 sealing attached to the second tube 163. The second port 169 includes a second valve configured to receive a gas provided to inflate the secondary cuff 112. The secondary cuff 112 is configured to inflate to block a trachea of a patient. The secondary cuff 112 operates independently from the distal cuff 109. The intubation device 100 can be used with the secondary cuff 112 deflated. For example, if the intubation device 100 is placed in the trachea for two lung ventilation, the distal cuff 109 alone may be inflated. Although the second channel 164 is not shown, it will be discussed in further detail.

The auxiliary conduit 115 is separate from the main lumen 106 and does not provide passage to the main lumen 106. The auxiliary conduit 115 is substantially smaller in diameter compared to the main lumen 106 and is not necessarily configured or intended for ventilation. In an embodiment, the auxiliary conduit 115 comprising an auxiliary tube 178 is sealingly connected to an auxiliary channel 181 formed in the tubular wall 104 of the flexible tubular body 103 and an auxiliary aperture 184 disposed between the distal cuff 109 and the secondary cuff 112. The auxiliary aperture 184 being an opening in the external surface of tubular wall 104 of the flexible tubular body 103 at an end of the auxiliary channel 181. The auxiliary channel 181 runs longitudinally within the wall 104 of the flexible tubular body 103 with an auxiliary aperture 184 in the wall 104 on the distal portion 121 that is located between the secondary cuff 112 and distal cuff 109. The auxiliary conduit 115 further comprises an auxiliary port 190 sealing attached to the auxiliary tube 178.

The auxiliary port 190 can be configured to receive a detachable cap 191. The auxiliary port 190 can be connected to a suction machine or other suitable devices or mechanisms. In an embodiment, the auxiliary port 190 can be configured to receive a detachable connection to a continuous positive airway pressure (CPAP) machine. In an embodiment, the auxiliary port 190 can be configured to receive a detachable connection (not shown) to an auxiliary device configured to aid in the specified procedure. The auxiliary conduit 115 can be used simultaneously while ventilation is supplied via the main lumen 106.

The main proximal connector 193 can be connected to the proximal end 127 of the flexible tubular body 103. The main proximal connector 193 can detachably connect to a mechanical ventilation device or other suitable devices or mechanisms. The intubation device 100 further comprises a first indicator 196 that circumscribes the flexible tubular body 103. The first indicator 193 is located at the distal portion 121 and is positioned between the distal cuff 109 and the auxiliary aperture 184. The intubation device 100 further comprises a second indicator 199 that circumscribes the flexible tubular body 103. The second indicator 199 is positioned between the proximal end 127 and the secondary cuff 112.

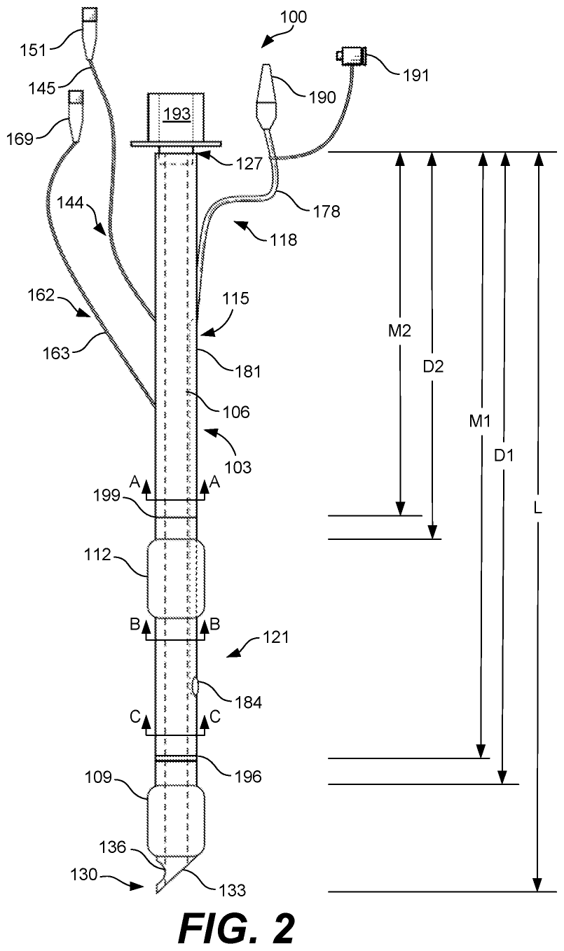

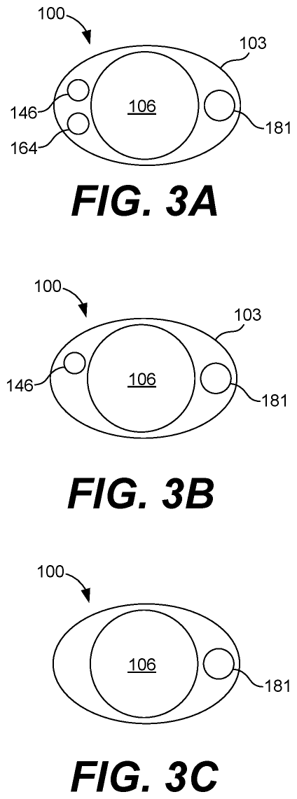

Illustrated in FIG. 2 is an example of an intubation device 100 for single or double lung ventilation in a straightened position to better indicate the relative position of features and cross-sections. As shown, the features correspond to the features of FIG. 1, with the distal cuff 109 and secondary cuff 112 shown inflated in FIG. 2. Sections A-A, B-B, and C-C are marked at positions along longitudinal length of the flexible tubular body 103. The relative position of the first channel 146, the second channel 164, and auxiliary channel 181 within the wall 104 of the flexible tubular body 103 will be shown in greater detail in FIGS. 3A-3C.

As previously discussed, the flexible tubular body 103 includes a wall 104 extending a longitudinal length (L) from a proximal end 127 to a distal end 130 forming the main lumen 106 with an inner diameter (ID). A distal portion 121 of the flexible tubular body 103 includes a bevel tip 133 open to the main lumen 106. The bevel tip 133 can be formed at the distal end 130 with a tapered angle relative to a longitudinal axis of the flexible tubular body 103. In an embodiment, a distal aperture 136 can be formed in the wall 104 opposite the bevel tip 133.

Shown as inflated in FIG. 2, the distal cuff 109 can be attached towards the distal portion 121 of the flexible tubular body 103 above the bevel tip 133 and the distal aperture 136. The distal cuff 109 can be inflated or deflated via a first conduit 144 in fluid communication with the distal cuff 109. The first conduit 144 comprises a first tube 145 sealingly connected to a first channel 146 (not shown) in the wall 104 of the flexible tubular body 103 at a first distance D1 from the proximal end 127. The first conduit 144 further comprises a first port 151 sealing attached to the first tube 145. The first port 151 having a first valve configured to receive a gas provided to inflate the distal cuff 109. In an embodiment, the distal cuff 109 can be inflated to block a bronchus of a patient.

The secondary cuff 112 can be formed from a tubular inflatable material. As shown, the secondary cuff 112 can be attached at a predetermined distance from the bevel tip 133 of the flexible tubular body 103. The secondary cuff 112 can be inflated or deflated via a second conduit 162 (not shown) in fluid communication with the secondary cuff 112. The second conduit 162 comprises a second tube 163 sealingly connected to a second channel 164 (not shown) in the wall 104 of the flexible tubular body 103 at a second distance D2 from the proximal end 127. The second conduit 162 further comprises a second port 169 sealing attached to the second tube 163. The second port 169 includes a second valve configured to receive a gas provided to inflate the secondary cuff 112. The secondary cuff 112 is configured to inflate to block a trachea of a patient. The secondary cuff 112 operates independently from the distal cuff 109. Although the second channel is not shown, it will be discussed in further detail.

The intubation device 100 further comprises a first indicator 196 that circumscribes the flexible tubular body 103. The first indicator 193 is located at the distal portion 121 and is positioned between the distal cuff 109 and the auxiliary aperture 184. The first indicator 196 can be configured to mark a first longitudinal distance M1 from the proximal end 127 of the flexible tubular body 103.

The intubation device 100 further comprises a second indicator 199 that circumscribes the flexible tubular body 103. The second indicator 199 is positioned between the proximal end 127 and the secondary cuff 112. The second indicator 199 can be configured to mark a second longitudinal distance M2 from the proximal end 127 of the flexible tubular body 103.

Turning to FIGS. 3A-C, representative embodiments of the relative position of the first channel 146, the second channel 164, and auxiliary channel 181 within the wall 104 of the flexible tubular body 103 are shown. Although one embodiment is shown, in FIGS. 3A-3C, alternative configurations can be envisioned to provide for the comfort of the patient. For example, to maintain substantially circular outer shape, the main lumen 106 may be off center and channels positioned differently.

In FIG. 3A, an embodiment of section A-A of FIG. 2 shows the cross-section of the flexible tubular body 103 above the secondary cuff 112. In this position, first channel 146, the second channel 164, and auxiliary channel 181 are shown within the wall 104 of the flexible tubular body 103. As shown in cross-section, the outer portion of the flexible tubular body is an oval shape with the main lumen 106 substantially circular and centered. The thickness of the wall 104 is configured to accommodate the channels 146,164,181 as shown. The first channel 146 and second channel 162 are shown on one side and the auxiliary channel 181 is shown on the other side.

In FIG. 3B, an embodiment of section B-B of FIG. 2 shows the cross-section of the flexible tubular body 103 below the secondary cuff 112 and above the auxiliary aperture 184. In this position, thickness of the wall 104 is configured to accommodate the first channel 146 and auxiliary channel 181 shown within the wall 104 of the flexible tubular body 103. Since the second channel 164 ends at the secondary cuff 109, the wall is filled at this section. The first channel 146 is shown on one side and the auxiliary channel 181 is shown on the other side.

In FIG. 3C, an embodiment of section C-C of FIG. 2 shows the cross-section of the flexible tubular body 103 below the auxiliary aperture 184 and above the distal cuff 109. In this position, thickness of the wall 104 is configured to accommodate only auxiliary channel 181 shown within the wall 104 of the flexible tubular body 103. The main shape of the flexible tubular body 103 is substantially the same, however, only the first channel 146 is shown on one side is shown to inflate or deflate the distal cuff 109.

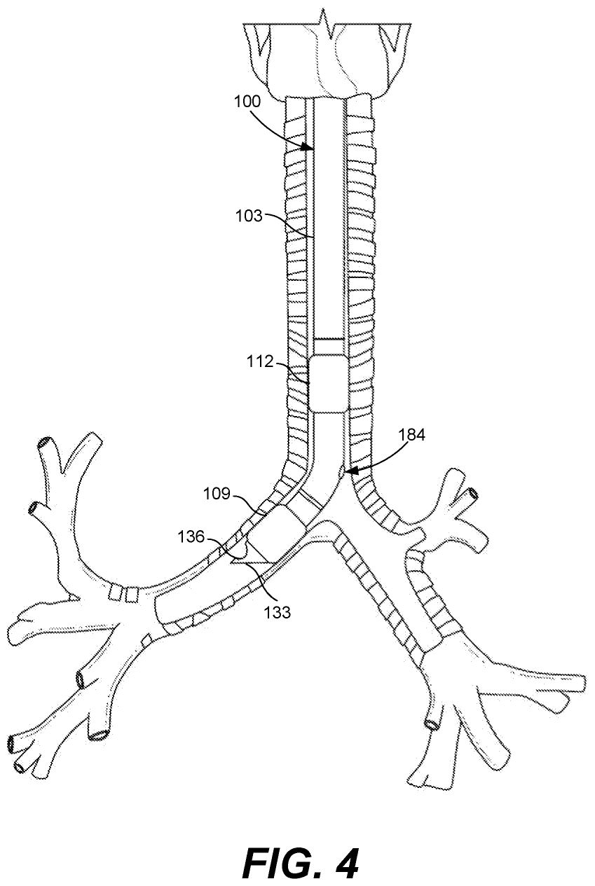

Turning to FIG. 4, an embodiment illustrating an example placement of the intubation device 100 is shown. In the example, the distal portion 121 of the intubation device 100 can be configured to extend into a bronchus of a patient. FIG. 4 is an illustration of the intubation device 100 positioned in a bronchus of a patient shown with both the distal cuff 109 and secondary cuff 112 inflated. In this example, the one lung is receiving mechanical ventilation via the main lumen 106 and the bevel tip 133 positioned in the right mainstem bronchus. Placement of the intubation device 100 can be facilitated using a fiberoptic bronchoscope (FOB). In an embodiment, the intubation device 100 further comprises a least one radio-opaque indicator (not shown) configured to mark the position of the radio-opaque indicator on a radiograph to confirm proper positioning of the intubation device 100. The auxiliary aperture 184 positioned between the two cuffs 109, 112 can be configured as a passive vent or suction port. In an embodiment, the non-ventilated lung can be allowed to deflate for surgical exposure during single-lung ventilation. In an embodiment, the non-ventilated lung can be allowed to receive passive oxygenation via the auxiliary aperture 184. In an embodiment, this can be a first position of the intubation device 100. For example, positioned during surgery for lung isolation and protection.

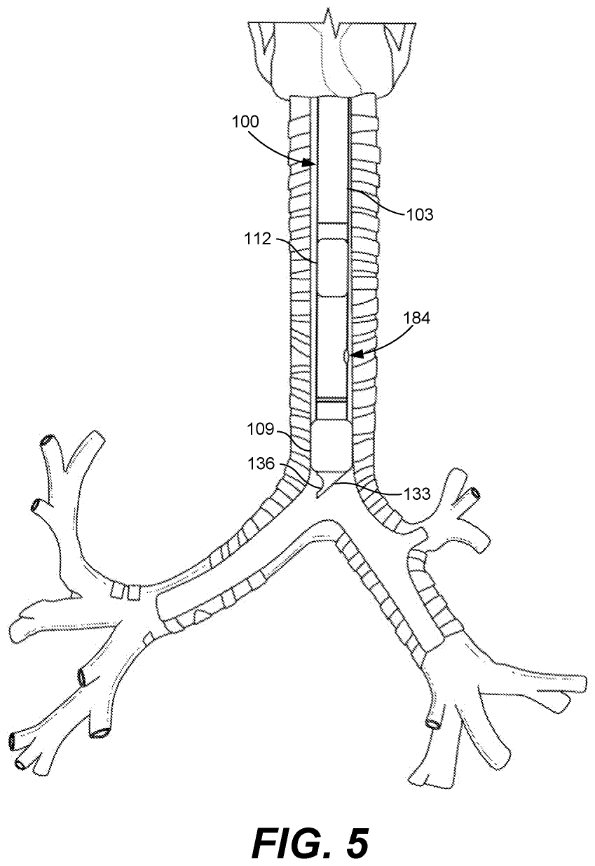

Turning to FIG. 5, an example illustrating the placement of the intubation device 100 is shown. In the example, the distal portion 121 of the intubation device 100 can be configured to extend into a trachea of a patient. FIG. 5 is an illustration of the intubation device 100 positioned in a trachea of a patient shown with only the distal cuff 109 inflated. In this example, both lungs are receiving mechanical ventilation via the main lumen 106. In an embodiment, the auxiliary aperture 184 can be an evacuation or suction port when the bevel tip 133 of the device is in the trachea, for suctioning of secretions above the inflated distal cuff 109. In an embodiment, this can be a second position of the intubation device 100. For example, without fully extubating, the intubation device 100 can allow a user to deflate the distal cuff 109 and secondary cuff 112 to reposition the device 100, then reinflate distal cuff 109 to provide ventilation to both lungs.

In an embodiment, an intubation method can be understood with respect to FIGS. 4 and 5 showing positions of the intubation device 100. The intubation method can include using a fiber optic bronchoscope to guide a bevel tip 133 of a distal end 130 of an intubation device 100 into a mainstem bronchus of a patient through the trachea (as shown in FIG. 4). The position of the distal end 130 of the intubation device 100 can be confirmed as in a bronchus of a patient visually, using the indicator markers 196,199, or by radiograph, using the radiopaque marker, or by other means. The distal cuff 109 can be inflated to secure the bevel tip 133 in the bronchus for single lung ventilation. The secondary cuff 112 can be inflated to protect a non-ventilated lung from aspiration. In an embodiment this may be a first position of the intubation device 100.

In an embodiment, continuous positive airway pressure can be delivered to the non-ventilated lung via an auxiliary channel 181 and an auxiliary aperture 184 within a wall 104 of the intubation device 100. In an embodiment, the auxiliary aperture 184 within the wall 104 of the intubation device 100 can be used to suction secretions with an external device via the auxiliary conduit 115.

Further to the method, the intubation device 100 can be repositioned to a second position (as shown in FIG. 5). From a position of the intubation device 100 placed in the bronchus, the method may further include deflating the distal cuff 109 and secondary cuff 112. Repositioning the intubation device 100 by withdrawing the bevel tip 133 from the bronchus into the trachea without fully extubating. Then confirming the position of the bevel tip 133 in the trachea of a patient visually, using the indicator markers 196,199, or by radiograph, using the radiopaque marker, or by other means. The distal cuff 109 can be inflated and both lungs receive ventilation.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

At least the following is claimed:

1. An intubation device, comprising:
a flexible tubular body having a wall extending a length from a proximal end to a distal end, the flexible tubular body forming a main lumen with an inner diameter, a distal portion having a bevel tip forming an opening to the main lumen, the bevel tip formed at the distal end with a tapered angle relative to a longitudinal axis of the flexible tubular body and a distal aperture formed in the wall opposite the opening;
a distal cuff comprising a tubular inflatable material, the distal cuff attached at the distal portion of the flexible tubular body above the bevel tip and the distal aperture;
a secondary cuff comprising a tubular inflatable material, the secondary cuff attached at a predetermined distance from the bevel tip of the flexible tubular body;
an auxiliary conduit comprising an auxiliary channel extending from a proximal portion of the flexible tubular body to an auxiliary aperture positioned between the distal cuff and secondary cuff and an auxiliary tube sealingly connected to the auxiliary channel in the wall at the proximal portion of the flexible tubular body; and
a main proximal connector connected to the proximal end of the flexible tubular body, the main proximal connector configured to detachably connect to a mechanical ventilation device.

2. The intubation device according to claim 1, further comprising:
a first tube sealingly connected to a first channel in the wall of the flexible tubular body at a first distance from the proximal end, the first tube and the first channel forming a first conduit in fluid communication with the distal cuff; and
a second tube sealingly connected to a second channel in the wall of the flexible tubular body at a second distance from the proximal end, the second tube and the second channel forming a second conduit in fluid communication with the secondary cuff.

3. The intubation device according to claim 2, further comprising:
a first port attached to the first tube, the first port having a first valve for inflation of the distal cuff; and
a second port attached to the second tube, the second port having a second valve for inflation of the secondary cuff.

4. The intubation device according to claim 1, further comprising a port attached to the flexible tubular body, the port configured to receive at least one of: a detachable cap, a connection to a suction machine, and a connection to a continuous positive airway pressure (CPAP) machine.

5. The intubation device according to claim 1, further comprising a first indicator that circumscribes the flexible tubular body, the first indicator located at the distal portion and positioned between the distal cuff and the auxiliary aperture, the first indicator marking a first longitudinal distance from the proximal end of the flexible tubular body.

6. The intubation device according to claim 5, further comprising a second indicator that circumscribes the flexible tubular body, the second indicator positioned between the proximal end and the secondary cuff, the second indicator marking a second longitudinal distance from the proximal end of the flexible tubular body.

7. The intubation device of claim 1, wherein the distal portion of the intubation device is configured to extend into a bronchus of a patient.

8. The intubation device of claim 1, wherein the distal portion of the intubation device is configured to be positioned in a trachea of a patient to provide ventilation to both lungs.

9. The intubation device according to claim 1, wherein the inner diameter of the main lumen is configured to slidingly receive a fiber optic bronchoscope (FOB).

10. The intubation device of claim 1, further comprising a least one radio-opaque indicator to mark a position on a radiograph for positioning the intubation device.

11. An intubation device, comprising:
a flexible tubular body having a wall extending a length from a proximal end to a distal end, the flexible tubular body forming a main lumen with an inner diameter, a distal portion having a bevel tip forming an opening to the main lumen, the bevel tip formed at the distal end with a tapered angle relative to a longitudinal axis of the flexible tubular body and a distal aperture formed in the wall opposite the opening;
a distal cuff comprising a tubular inflatable material, the distal cuff attached at the distal portion of the flexible tubular body above the bevel tip and the distal aperture;
a secondary cuff comprising a tubular inflatable material, the secondary cuff attached at a predetermined distance from the bevel tip of the flexible tubular body;
a first conduit in fluid communication with the distal cuff, the first conduit comprising a first channel formed in the wall extending from a first specified position in a proximal portion of the flexible tubular body to a first inflation aperture configured to inflate or deflate the distal cuff and a first tube sealingly connected to the first channel in the wall at the first specified position in the proximal portion of the flexible tubular body;
a second conduit in fluid communication with the secondary cuff, the second conduit comprising a second channel formed in the wall extending from a second specified position in a proximal portion of the flexible tubular body to a second inflation aperture configured to inflate or deflate the secondary cuff and a second tube sealingly connected to the second channel in the wall at the second specified position in the proximal portion of the flexible tubular body;
a third conduit comprising an auxiliary channel formed in the wall extending from a third specified position in a proximal portion of the flexible tubular body to an auxiliary aperture positioned between the distal cuff and secondary cuff and an auxiliary tube sealingly connected to the auxiliary channel in the wall at the specified position in the proximal portion of the flexible tubular body; and
a main proximal connector connected to the proximal end of the flexible tubular body, the main proximal connector configured to detachably connect to a mechanical ventilation device.

12. The intubation device according to claim 11, further comprising:
a first port attached to the first tube, the first port having a first valve for inflation of the distal cuff; and
a second port attached to the second tube, the second port having a second valve for inflation of the secondary cuff.

13. The intubation device according to claim 12, further comprising a port attached to the flexible tubular body, the port configured to receive at least one of: a detachable cap, a connection to a suction machine, and a connection to a continuous positive airway pressure (CPAP) machine.

14. The intubation device according to claim 11, further comprising:
first and second indicators that circumscribe the flexible tubular body, wherein
the first indicator is located at the distal portion and positioned between the distal cuff and the auxiliary aperture, the first indicator configured to mark a first longitudinal distance from the proximal end of the flexible tubular body, and
the second indicator positioned between the proximal end and the secondary cuff, the second indicator configured to mark a second longitudinal distance from the proximal end of the flexible tubular body.

15. The intubation device according to claim 11, wherein the internal diameter of the main lumen ranges from about 4.0 mm to about 13.5 mm.

16. An intubation method for lung isolation and protection, comprising:
using a fiber optic bronchoscope to guide a bevel tip of a distal end of an intubation device into a mainstem bronchus of a patient through the trachea;
confirming the position of the distal end of an intubation device in a bronchus through the trachea of a patient;
inflating a distal cuff to secure the bevel tip in the bronchus for single lung ventilation; and
inflating a secondary cuff to protect a non-ventilated lung from aspiration.

17. The intubation method according to claim 16, further comprising:
delivering continuous positive airway pressure to the non-ventilated lung via an auxiliary channel and an auxiliary aperture within a wall of the intubation device.

18. The intubation method according to claim 16, further comprising:
suctioning secretions via an auxiliary aperture within the wall of the intubation device.

19. The intubation method according to claim 16, further comprising:
deflating the distal cuff;
deflating the secondary cuff;
repositioning the intubation device by withdrawing the bevel tip from the bronchus into the trachea without full extubation;
confirming the position of the bevel tip in the trachea;
inflating the distal cuff in the trachea; and
ventilating both lungs.

20. The intubation method according to claim 16, further comprising:
confirming the position of the bevel tip by a least one radio-opaque indicator configured to mark the position of the radio-opaque indicator on a radiograph.
